(19) United States
(12) Patent Application Publication
(10) Pub. No.: US 2016/0228282 A1
(43) Pub. Date: Aug. 11, 2016

(54) OSTOMY APPLIANCE

(71) Applicant: Georgann M. Carrubba, Batavia, NY (US)

(72) Inventor: Georgann M. Carrubba, Batavia, NY (US)

(21) Appl. No.: 14/924,496

(22) Filed: Oct. 27, 2015

Related U.S. Application Data
(63) Continuation-in-part of application No. 14/617,460, filed on Feb. 9, 2015.

(51) Int. Cl. A61F 5/443; A61F 5/441; A61F 5/44; A61F 5/448

(52) U.S. Cl. CPC A61F 5/443; A61F 5/448; A61F 5/441; A61F 5/4404

(57) ABSTRACT

An ostomy appliance having a releasable receptacle for concealing an ostomy bag where the receptacle is asymmetrical and includes an enlarged cavity portion located below the stoma which provides additional area into which the bag may unroll as waste collects therein. In an alternate embodiment there is no bag and waste collects directly into the receptacle.

### BACKGROUND OF THE INVENTION

The present invention relates to ostomy appliances. More particularly, the present invention relates to an ostomy appliance which is designed to provide a high level of discreteness to the user on an as needed basis.

An “ostomy” is the generic term for a surgical procedure such as the ileostomy, colostomy and urostomy which leave the patient with a digestive waste opening formed by attaching the patient's intestine (small intestine in the case of an ileostomy; large intestine in the case of a colostomy) to the perimeter of an incision made in the abdominal wall. This part of the intestine is surgically opened to form what is called the “stoma” wherethrough digestive waste exits the body. The procedure may be reversible or irreversible depending on the circumstances. It is estimated that up to 750,000 Americans have an ostomy.

A digestive waste collection bag is attached about the stoma to collect the waste existing therefrom. The collection bag and associated body attachment parts are commonly referred to as the ostomy appliance or assembly. Ostomy appliances are offered in single piece and two piece systems. In the single piece system, the bag has an opening which is aligned with an opening in and then permanently fixed to a disc or flange which has an adhesive surface opposite the bag which secures the flange and bag to the body about the stoma. In the two-piece system, the bag is detachably secured to the flange, commonly via a snap ring on the flange which fits into a cooperatively formed groove attached to the perimeter of the bag opening. In either the single or two piece systems, the bag typically includes an open end opposite the flange end which may be alternately open and closed using a clamp. The bag is clamped closed during use and opened to empty the waste contents from the bag. The bag may be cleaned and reused or discarded with a new bag being attached to the flange.

Users of ostomy appliances often complain about the inconveniences of the appliance including lack of discreteness. For example, there is no control over when waste deposits into the bag which may thus fill and noticeably expand beneath the clothing at inconvenient times, inevitably causing embarrassment to the user. Attempts to address this problem have been at best a minor improvement while others appear to pose the threat of actual physical harm to the user by applying a positive pressure against the stoma.

One example of an ostomy appliance which addresses the above drawbacks in the prior art is shown and described in commonly owned U.S. Pat. No. 8,657,799, the disclosure of which is incorporated herein by reference. The '799 patent provides an ostomy appliance which is compact and has a receptacle portion which accommodates a certain amount of waste before it must be opened and emptied. For users that desire to wear the ostomy appliance a bit longer than the '799 receptacle may allow, there remains a need for an ostomy appliance having a receptacle designed to accommodate a bit more waste. This would allow the user to wear the appliance for a longer time prior to opening and emptying yet still provide enhanced discreteness during periods of physical activity and close personal encounters, all without application of potentially dangerous pressures on the stoma as occurs in the prior art. Additionally, to aid in maintaining discreteness, the appliance may include a sensor within the receptacle or bag of the appliance which is capable of wirelessly communicating with an external device, such as a cell phone, when the bag is full or nearly full. Furthermore, to improve hygiene, the bag may be constructed of a biodegradable, and in one aspect, of a flushable material, thereby obviating the need for emptying and cleaning the bag after or between uses.

### SUMMARY OF THE INVENTION

The present invention provides an ostomy appliance and method which includes a cup-shaped receptacle having an attachment mechanism about the open perimeter thereof which may be removably secured to a mating attachment mechanism (e.g., an ostomy flange) secured to the adhesive patch which includes a central opening for locating about the stoma opening on the user's body. The bag includes a ring element at the open stoma-facing end thereof which removably attaches to a mating ring element located around the stoma opening and radially inwardly of the receptacle attachment mechanism on the patch. When it is desired to contain the bag for increased discreteness, the user gathers or rolls the empty bag up upon itself into a rolled position, positions the receptacle over the bag and then secures the cooperative attachment mechanism together thereby securing the receptacle to the ostomy flange with the bag enclosed therein in the rolled condition.

The receptacle may have one or more vent holes formed therein to allow gas to escape therefrom. A vent cap may be provided which is normally closed but will automatically open upon a rise in pressure inside the receptacle to allow the egress of gas from the receptacle. Once the pressure is relieved the vent cap automatically closes. A covering may be placed over the receptacle to provide further discreteness and comfort to the user.

The receptacle includes an enlarged internal cavity which, when attached to the ostomy flange on the user's body, preferably extends downwardly in line with the length of the body toward the user's feet. This enlarged cavity portion of the receptacle provides portion within which the bag may deploy (e.g., unroll) as the bag fills with the user's waste. This particular embodiment thus allows the receptacle to remain in place for a longer time than a receptacle that has a smaller receptacle cavity size. In a preferred embodiment, the receptacle is asymmetrically shaped with the smaller side of the receptacle located above the stoma. This asymmetrical shape helps minimize the size of the receptacle while still proving added cavity portion where it is most useful. More particularly, the side of the receptacle which is smaller extends above the stoma where waste is not normally directed and the enlarged area is located on the opposite side of the receptacle where the waste and bag are directed by gravity when the user is sitting upright or standing.

When the bag is held in the receptacle, waste will deposit therein the usual manner. As waste enters the bag, the bag will begin to expand and unroll slightly within the confines of the receptacle. There is thus no positive pressure being applied to the stoma and waste is allowed to naturally exit the user's stoma and enter the bag. It is not intended that the receptacle be in place over the bag for more than a few hours at a time and it is therefore not expected that waste will be prevented from naturally exiting the stoma for any potentially harmful extended period of time. Rather, the receptacle is only intended to be in place during times when the user would like an increased level of discreteness and control over waste evacuation into the bag. Once the bag is full and/or the receptacle is no longer needed, the user simply detaches the receptacle from the flange whereupon the bag is allowed to fully unroll and is either left to completely fill or emptied and cleaned or replaced with a new bag, as desired.

In another embodiment, the bag is not used and waste collects directly into the receptacle which may be removed, emptied of waste, cleaned and reattached as desired. This provides an option to the user who may not want to use a bag or when their bag supply has run out.

In a further embodiment, the receptacle or bag includes a sensor configured to sense whether the bag is full or nearly full. The sensor may be in wireless connectivity with an external device, such as a cell phone, which alerts the user of the bag's condition at which point the user can empty to bag as necessary.

In still a further embodiment, the bag may be constructed of a biodegradable material, and still further may be constructed of a flushable material. To that end, the bag may be a single-use bag which may be disposed of in the trash or flushed down the toilet after use. A new bag can then be installed. In this manner, user hygiene is improved as emptying, cleaning and reusing of the bag is no longer required.

In yet a further embodiment, the bag may include a liquid absorbent material so as to reduce the potential for spills, as well as to ease emptying and cleaning of used bags. In one aspect, a desiccant paper product may line the inner surface of the bag. Additionally or alternatively, the bag may include an absorbent powder, such as a super-absorbent polymer packet.

### BRIEF DESCRIPTION OF THE DRAWING

The above-mentioned and other features and advantages of this invention, and the manner of attaining them, will become apparent and be better understood by reference to the following description of the invention in conjunction with the accompanying drawing, wherein:

FIG. 1 is a perspective view of an embodiment of the inventive ostomy device;

FIG. 2 is a side elevational view of the ostomy device of FIG. 1 showing the covering in position over the ostomy bag container with parts thereof shown in section;

FIG. 3 is an exploded perspective view of the ostomy device absent the ostomy bag;

FIG. 4 is a perspective view showing the ostomy bag attached to the stoma flange with the bag in the unfolded position;

FIG. 5 is the view of FIG. 4 showing the ostomy bag in the folded condition and the ostomy bag container in portioned relation thereto;

FIG. 6 is the view of FIG. 5 showing the ostomy bag container attached to the stoma flange with the folded bag showing in dotted lines inside the container; and

FIG. 7 is a side elevational view of an alternative embodiment of an ostomy device.

### DETAILED DESCRIPTION OF A PREFERRED EMBODIMENT

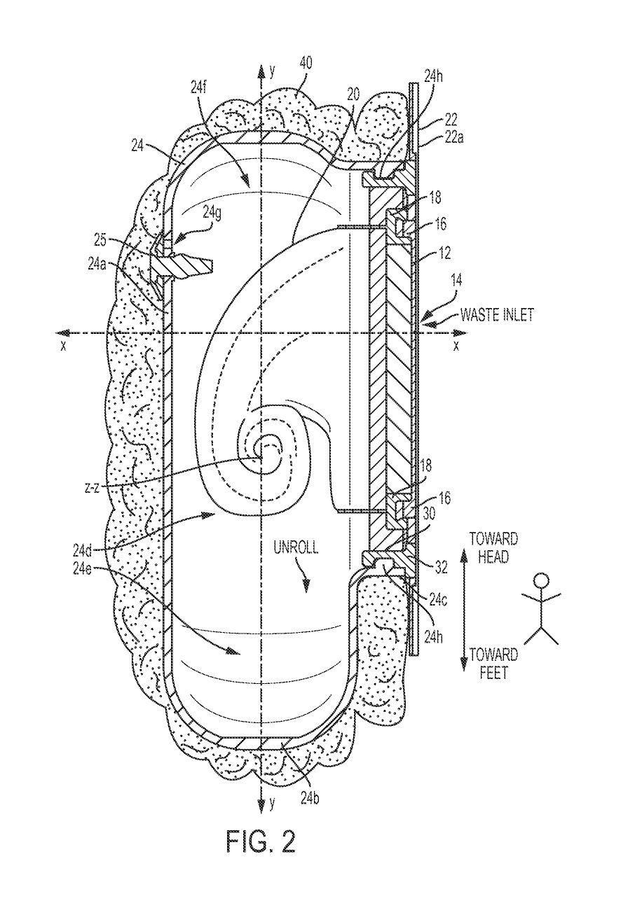

Referring now to the drawing, there is seen in the various figures an ostomy appliance according to one embodiment of the invention indicated generally by the reference numeral 10. Ostomy appliance 10 includes a disc 12 having a central opening 14 for aligning with and positioning over a user's stoma (not shown). Disc 12 includes an adhesive surface 12a (see FIG. 2) for securing the disc about the stoma body tissue. Release paper (not shown) protects the adhesive surface 12a until time of use whereupon the user removes the release paper and adheres the disc surface 12a onto the stoma tissue with the opening of the stoma aligned with the disc opening 14.

A ring-shaped groove element 18 is attached to the perimeter of a first opening 20a of waste collection bag 20 and a ring-shaped flange 16 is affixed to disc 12 such that the bag may be releasably attached to the disc 12. Skirt 22 extends outwardly from ring-shaped flange 16 and includes an adhesive surface 22a for adhering to the user's body tissue which surrounds the stoma. A release paper (not shown) may protect the adhesive surface 22a until time of use whereupon the user removes the release paper and adheres the skirt 22 to the user's body while also adhering the disc adhesive surface 12a to the adjacent stoma tissue.

A cup-shaped receptacle 24 is provided for times when the user desires an increased level of discreteness. Receptacle 24 includes a bottom wall 24a and a side wall 24b terminating in a top perimeter edge 24c all defining an interior cavity 24d. The shape of the receptacle is such that side wall 24b forms an enlarged cavity portion 24e which extends downwardly in line with the length of the body toward the legs when attached to the user in the intended manner. This enlarged cavity portion 24e of the receptacle provides more portion within which the bag 20 may fit and begin to expand or unroll as the bag fills with the user's waste. This particular embodiment thus allows the receptacle to remain in place for a longer time than a receptacle that is more compact.

As seen best in FIG. 2, receptacle 24 is asymmetrically shaped about a horizontal axis x-x (relative to vertically standing user) extending through the center of opening 14 which is substantially concentrically placed over the user's stoma. The receptacle smaller cavity portion 24f is located above axis x-x and the stoma (toward user's head) and the enlarged cavity portion 24e is located below axis x-x and the stoma (toward the user's feet). This asymmetrical shape helps minimize the overall size of the receptacle in that the smaller cavity portion 24f is located above the stoma where waste does not usually get directed and the enlarged cavity portion 24e is located below the stoma where the waste and bag 20 are directed within the confines of the receptacle 24 due to gravity when the user is sitting upright or standing. In this regard, it is preferred that the user roll bag 20 up about an axis z-z which extends in the horizontal plane of and perpendicular to axes x-x and longitudinal axis y-y. As such, the bag 20 will be able to freely unroll in the direction of enlarged cavity portion 24e along and parallel to vertical longitudinal axis y-y as it is filled with waste.

Receptacle bottom wall 24a may include a small aperture 24g to allow gases from the stoma to vent therethrough. A vent plug 25 is provided which is operable to move between a normally closed aperture position and an open aperture position where a rise in gas pressure within receptacle 24 above a threshold pressure causes vent plug 25 to move to the open aperture position and thereby allowing the egress of gas from receptacle 24 through aperture 24g. Once the gas is released and the receptacle internal pressure lowers below the threshold pressure, the vent plug automatically sits back over and closes aperture 24g. This allows intermittent gas release rather than constant gas release and also prevents an unsafe pressure build-up within the receptacle.

A receptacle attachment mechanism is provided for releasably securing receptacle 24 to the disc 12/flange 22 combination. The receptacle attachment mechanism may be provided in the form of a luer-type mechanism, for example. In this preferred embodiment, receptacle top perimeter edge 24c includes one or more tabs 24h which align with and may be screwed into a respective number of curved slots 30 formed in outer ring 32 affixed to disc 12 and thereby releasably securing receptacle 24 to ring 32. Prior to securing receptacle 24 to ring 32, the user rolls bag 20 up upon itself as shown in FIG. 5. Once receptacle 24 is attached to ring 32 in the manner described, bag 20 is located within receptacle 24 as shown in FIG. 6. Although a Luer-type mechanism is described and shown herein as comprising the receptacle attachment mechanism, it is of course understood that other attachment mechanisms which releasably secure receptacle 24 to ring 32 as desired.

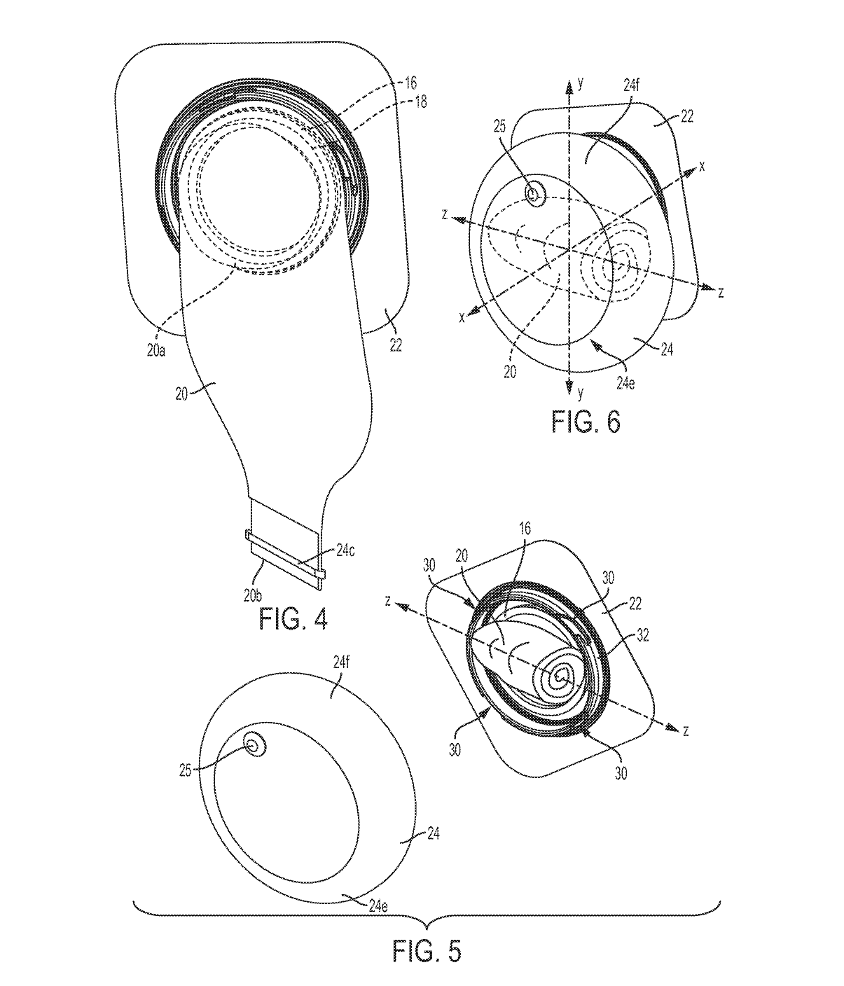

As described above, waste is free to enter bag 20 which may expand and unroll in the direction of enlarged cavity portion 24e. There is no positive pressure applied to the stoma during this time. The user may cover receptacle 24 with a fabric covering 40 having an elasticized opening 40a as seen in FIG. 2. In this manner, the user has increased his/her level of discreteness by confining their ostomy bag 20 to a relatively small receptacle 24 with a soft fabric covering. The user may thus engage in physical or other social activities with a greater sense of discreteness due to the control and concealment of the ostomy bag afforded by receptacle 24. When the user removes receptacle 24, bag 20 is free to fully unroll whereupon the user may empty waste from second bag opening 20b using resealable closure 20c (FIG. 4).

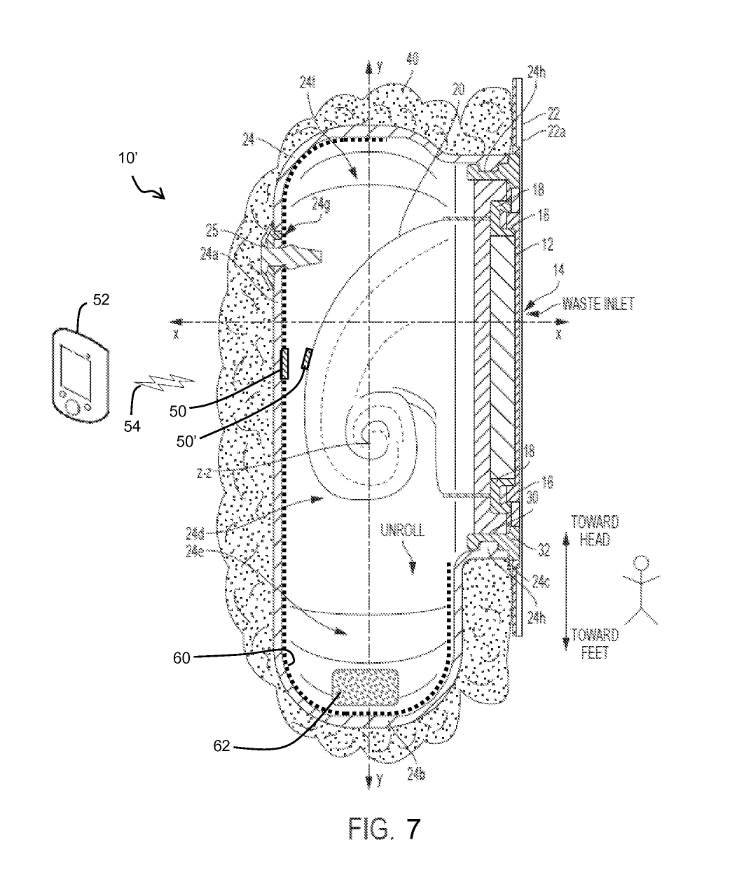

Turning now to FIG. 7, a further embodiment of an ostomy appliance 10′ may include a sensor 50 configured to sense when bag 20 and/or receptacle 24 is full or near full so as to require emptying or changing of bag 20. Sensor 50 may be, for instance and without specifically limited thereto, a pressure sensor that detects pressure upon the sensor as bag 20 expands within receptacle 24 as it is being filled. Placing sensor 50 at or near horizontal axis x-x may permit bag 20 to more fully occupy interior cavity 24d and enlarged cavity portion 24e before pressure is exerted upon sensor 50. In this manner, an optimal capacity of bag 20 may be utilized before the filled bag is replaced. Sensor 50 may further include circuitry and software needed to provide a wireless signal 54 to an external device, such as a cell phone 52. Wireless signal 54 may then discretely inform the user of the need to replace bag 20. In an alternative embodiment a sensor 50′ may located on the bag itself such that as the bag fills, the sensor is triggered so as to emit wireless signal 54. It should be noted that while sensors 50/50′ have been described as a pressure sensor, any suitable sensor may be utilized and such other and additional sensors are to be considered within the present disclosure.

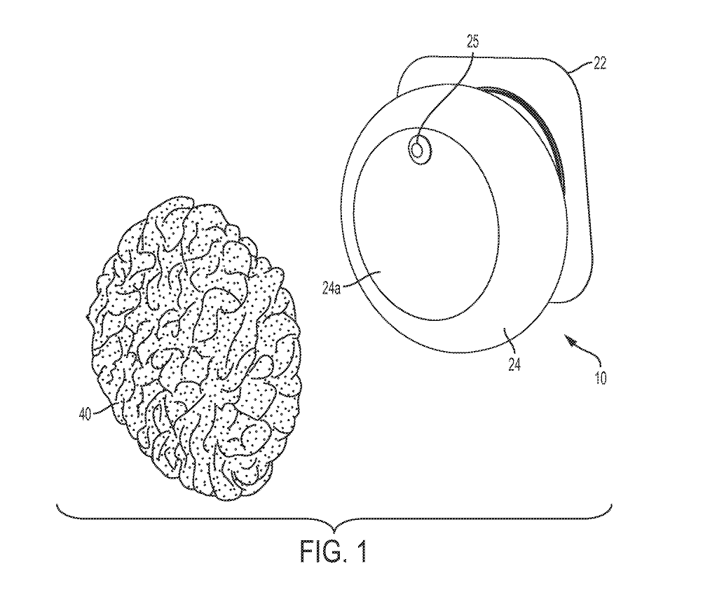

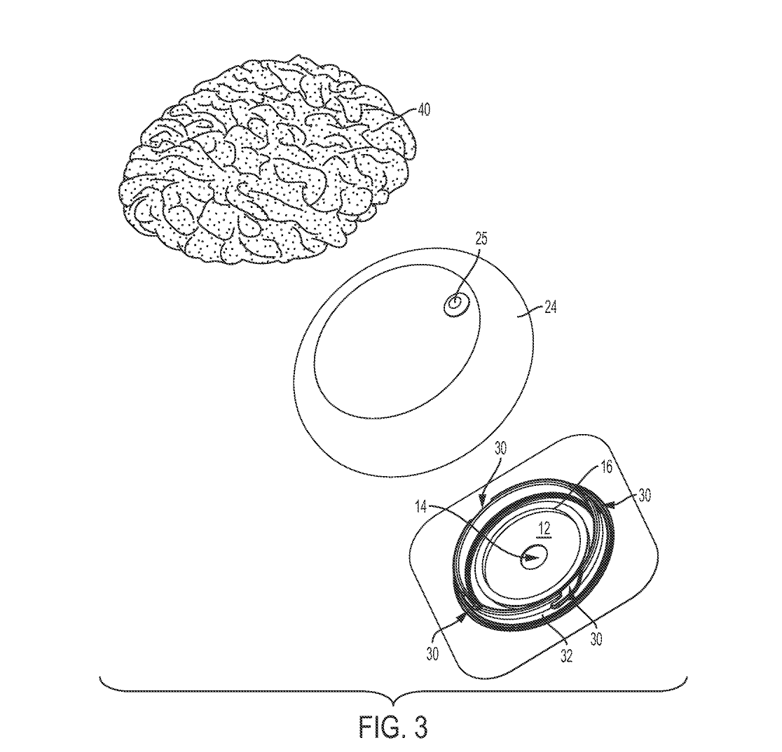

As further shown in FIG. 7, appliance 10′ may further include a liquid absorbent material to prevent liquid spills or collect liquid should a spill occur. In one aspect, receptacle 24 may be line with a liquid absorbent paper 60. One possible example of a suitable paper would be from IMPAK Corporation, Los Angeles, California, including but not limited to its Grade 460 paper comprised of silica gel impregnated cellulose fibers. Additionally or alternatively, receptacle 24 may be configured to include a liquid absorbent desiccant material—either a loose powder or a powder housed within a liquid dissolvable packet, such as packet 62. One possible example of a suitable packet would be IMPAK Corporation's Liquasafe packets comprising sodium polyacrylate which can absorb up to 300 times its weight in aqueous solutions. While paper liner 60 and packet 62 are shown and described with reference to appliance 10′, it should be understood by those skilled in the art that appliance 10 (FIGS. 1-6) may also be similarly equipped with liquid absorbent material. Further, while shown and described as being placed within receptacle 24, the desiccant may also be provided within bag 20.

In another embodiment, the bag 20 is not used and waste collects directly into the receptacle 24 and will drop by gravity into enlarged cavity portion 24e. Receptacle 24 may thereafter be removed, emptied of waste, cleaned and reattached to disc 12 as desired. This provides an option to the user who may not want to use a bag or when their bag supply has run out.

Other embodiments of the invention may include features such as making receptacle 24 of a color changing material which is activated with increased heat. When waste is depositing into the bag or directly into the receptacle, the heat of the waste will heat the receptacle which in turn will change color, altering the user that it may be time to check their ostomy bag or receptacle or possible emptying or changing.

Still further embodiments of the invention may include a bag 20 which is constructed of a biodegradable material. In this manner, when bag 20 is full or nearly full, the user can remove the bag as described above; but rather than emptying and cleaning the bag, the user can deposit the bag in the trash and install a new bag 20 as described above. Moreover, bag 20 may be constructed of a flushable material such that the filled bag need not be deposited in the trash, but may be flushed down the toilet. In either case, biodegradable/flushable bag 20 improves user hygiene by removing the need to empty, clean and reinstall bag 20. As such, bag 20 may be a single-use bag where the user only uses each bag 20 once. In this manner, the risk for infection is reduced as the user is no longer inadvertently handling the contents of soiled bags and is no longer reinstalling used bags on the appliance as is known in the art.

What is claimed is:

1. An ostomy device, comprising:
a) a disc having an adhesive surface for applying to stoma tissue;
b) a ring-shaped flange attached to said disc on the surface thereof opposite said adhesive surface;
c) a bag having a first opening having a ring-shaped groove attached to the perimeter of said first opening, said ring-shaped groove releasably attachable to said ring-shaped flange to releasably attach said bag to said disc;
d) an asymmetrically shaped receptacle having a bottom wall, side wall and a top perimeter edge defining an interior cavity having an enlarged cavity portion;
e) a receptacle releasable attachment mechanism configured to releasably attach said receptacle to said disc.

2. The ostomy device of claim 1 and further including an outer ring affixed to said disc and wherein said releasable attachment mechanism comprises one or more tabs formed on said receptacle top perimeter edge which may releasably attach to a like number of curved slots formed in said outer ring.

3. The ostomy device of claim 1 and further including an aperture in said receptacle bottom wall and a vent plug located adjacent said aperture and operable to move between a normally closed aperture position and an open aperture position where a rise in gas pressure within said receptacle above a threshold pressure causes said vent plug to move to said open aperture position and thereby allowing the egress of gas from said receptacle through said aperture.

4. An ostomy device, comprising:
a) a disc having an adhesive surface for applying to stoma tissue;
b) a ring-shaped flange attached to said disc on the surface thereof opposite said adhesive surface;
c) an asymmetrically shaped receptacle having a bottom wall, side wall and a top perimeter edge defining an interior cavity having an enlarged cavity portion;
e) a receptacle releasable attachment mechanism configured to releasably attach said receptacle to said disc.

5. The ostomy device of claim 1 wherein said receptacle includes a color changing material which is activated by heat.

6. A method of using an ostomy appliance, said method comprising the steps of:
a) providing an ostomy flange having a disc and an adhesive surface and a central opening;
b) releasably attaching said disc to a user via said adhesive surface with said central opening located over the user's stoma;
c) providing an asymmetrical receptacle having a bottom wall, side wall and open perimeter top all defining an interior cavity having an enlarged cavity portion; and
d) removably attaching said receptacle to said disc with said open top perimeter positioned over said disc central opening and stoma.

7. The method of claim 6 and further comprising the step of orienting said receptacle with said enlarged cavity portion extending in the direction of the user's feet.

8. The method of claim 6 and further comprising the step of providing a bag having an open top and removably attaching said bag to said disc with said bag top opening positioned over said central opening and stoma.

9. The method of claim 7 and further comprising the step of rolling said bag up on itself about an axis which extends perpendicular to the longitudinal axis of said receptacle.

10. The method of claim 6 and further comprising the steps of:
a) providing an outer ring with one or more curved slots formed therein and affixing said outer ring to said disc;
b) providing one or more tabs on said receptacle top perimeter edge; and
c) releasably attaching said receptacle to said outer ring with said one or more tabs engaging said one or more curved slots, respectively.
